(19) United States
(12) Patent Application Publication
(10) Pub. No.: US 2023/0056702 A1
(43) Pub. Date: Feb. 23, 2023

(54) REDUCING OXYGEN LEVELS IN THE GASTROINTESTINAL TRACT

(71) Applicant: Northeastern University, Boston, MA (US)

(72) Inventors: Kim LEWIS, Newton, MA (US); Norman PITT, Orinda, CA (US); Madeleine MORRISSETTE, Arlington, MA (US)

(73) Assignee: Northeastern University, Boston, MA (US)

(21) Appl. No.: 17/821,644

(22) Filed: Aug. 23, 2022

Related U.S. Application Data
(60) Provisional application No. 63/235,857, filed on Aug. 23, 2021.

(51) Int. Cl. A61K 35/74; A61K 35/34; A61K 36/81; A61K 36/21; A61K 36/064; A61K 36/062; A61K 35/68; A61K 36/05; A61P 1/04

(52) U.S. Cl. CPC A61K 35/74; A61K 35/34; A61K 36/81; A61K 36/21; A61K 36/064; A61K 36/062; A61K 35/68; A61K 36/05; A61P 1/04

(57) ABSTRACT

The present disclosure is directed towards a method of treating a disease or condition associated with gut inflammation. The present disclosure comprises administering, to a patient in need thereof, a composition comprising oxygen-scavenging membrane fragments or membrane vesicles. In some embodiments, membrane vesicles are derived from bacteria. In some embodiments, membrane vesicles are derived from the cytoplasmic membranes of Escherichia coli, Salmonella typhimurium, Gluconobacter oxydans, Pseudomonas aeruginosa, or Acetobacter.

### RELATED APPLICATIONS

This application claims the benefit of priority to U.S. Ser. No. 63/235,857, filed on Aug. 23, 2021, the entire disclosure of which is hereby incorporated by reference.

### FIELD OF THE INVENTION

The invention relates generally to a method of treating or preventing a disease which is alleviated by depleting oxygen levels in the gut.

### BACKGROUND OF THE INVENTION

A healthy gut is largely anaerobic. The mucosa prevents oxygen from diffusing into the gut while intestinal epithelial cells respire, reducing the gut lumen. Accordingly, human commensal bacteria are commonly anaerobic. Many pathobionts, undesirable bacteria associated with disease, can live under both anaerobic and aerobic conditions. When the functionality of epithelial cells decreases, for example with age, or the colon gets inflamed, oxygen leaches into the gut, fueling the propagation of pathobiotints. This exacerbates conditions such as inflammatory bowel disease (IBD).

IBD is an umbrella term used to describe disorders that involve chronic inflammation of the digestive tract. Types of IBD may include ulcerative colitis, which involves inflammation and sores (ulcers) along the superficial lining of the large intestine (colon) and rectum, and Crohn's disease, which is characterized by inflammation of the lining of the digestive tract, which often can involve the deeper layers of the digestive tract. Both ulcerative colitis and Crohn's disease usually are characterized by diarrhea, rectal bleeding, abdominal pain, fatigue and weight loss. Up to three million Americans have some form of IBD. The condition affects all ages and genders.

People with IBD have a higher risk of developing colon (colorectal) cancer. Other potential complications include anal fistula (a tunnel that forms under the skin connecting an infected anal gland and the anus), anal stenosis or stricture (narrowing of the anal canal where stool leaves the body), anemia (low levels of red blood cells) or blood clots, kidney stones, liver disease, such as cirrhosis and primary sclerosing cholangitis (bile duct inflammation), malabsorption and malnutrition (inability to get enough nutrients through the small intestine), osteoporosis, perforated bowel (hole or tear in the large intestine), and toxic megacolon (severe intestinal swelling).

It would be desirable to identify compositions and methods that reduce the oxygen level in the gut, which in turn, lowers the chance of gut inflammatory disease.

### SUMMARY OF THE INVENTION

The disclosure provides a method of treating a disease or condition associated with gut inflammation by administering a therapeutically effective amount of a composition comprising oxygen-scavenging membrane fragments and/or membrane vesicles, or a pharmaceutically acceptable salt, solvate, hydrate, cocrystal, or prodrug thereof.

In some embodiments, the oxygen-scavenging membrane fragments and/or membrane vesicles are derived from bacteria.

In some embodiments, the oxygen-scavenging membrane fragments and/or membrane vesicles are derived from the group consisting of cytoplasmic membranes of Escherichia coli, Salmonella typhimurium, Gluconobacter oxydans, Pseudomonas aeruginosa, Acetobacter, Lactobacillus or Bacillus.

In some embodiments, the sources of the oxygen-scavenging membranes are selected from the group consisting of beef heart muscle, potato tuber, spinach, Saccharomyces, Neurospora, Aspergillus, Euglena, and Chlamydomonas.

In some embodiments, the composition contains the oxygen-scavenging membrane fragments in the amount of about 0.001 units/mL to about 100 units/mL, from about 0.01 units/mL to about 10 units/mL, from about 0.3 unit/mL to about 10 units/mL, or from about 1 unit/mL to about 10 units/mL.

In some embodiments, the composition is administered as a parabiotic to prevent inflammatory bowel disease development. In some embodiments, the composition is taken to maintain microbiome diversity. In some embodiments, the composition administered is OXYRASE.

In some embodiments, the disease or condition is selected from the group consisting of inflammatory bowel disease, traveler's diarrhea, Crohn's disease, ulcerative colitis, celiac disease, polyps, cancer, celiac disease, diverticulitis, malabsorption, short bowel syndrome, gastritis, gastroparesis, gastroenteritis, peptic ulcers, and intestinal ischemia.

In some embodiments, the composition contains a cryoprotectant in an amount of about 15% by weight to about 65% by weight, or from about 50 wt % to about 65 wt %. In some embodiments, wherein the cryoprotectant is selected from the group comprising glycerol, dimethyl sulfoxide (DMSO), and glycols, including ethylene glycol, propylene glycol, polyethylene glycol, and glycerol.

In some embodiments, the composition further comprises a hydrogen donating substance in the amount of about 0.1 wt % to about 5 wt %.

In some embodiments, the oxygen-scavenging membrane fragments and/or membrane vesicles are derived from mitochondria that reduce oxygen to water in the presence of a hydrogen donating substance. In some embodiments, the donating substance is selected from the group comprising lactic acid, succinic acid, alpha-glycerol phosphate, formic acid, malic acid, pyruvic acid and the corresponding salts thereof.

In some embodiments, wherein the composition may be in the form selected from the group comprising an injection, a solution, a suspension, or emulsion. In some embodiments, the composition is administered orally. In some embodiments, the composition is administered through a rectal route. In some embodiments, the composition is administered intracolonically.

### BRIEF DESCRIPTION OF THE FIGURES

The foregoing summary, as well as the following detailed description of embodiments of the disclosure, will be better understood when read in conjunction with the appended drawings and figures.

The patent or application file contains a least one drawing executed in color. Copies of this patent or patent application publication with color drawing(s) will be provided by the Office upon request and payment of the necessary fee.

FIGS. 1A-1B: FIG. 1A is a graph showing weight (g) of mice throughout the duration of DSS experiment. FIG. 1B is a graph showing percent of initial body weight of mice on Day 7, 24-hours after DSS removal.

FIGS. 2A-2B: FIG. 2A is a graph showing colon length (cm) at sacrifice. FIG. 2B is a graph showing CFU Enterobacteriaceae/g of cecal contents determined by plating cecal contents on MacConkey agar.

FIGS. 3A-3C: FIG. 3A is a graph showing percent of initial body weight of mice. FIG. 3B is a graph showing percent of body weight of mice on Day 8. FIG. 3C is a graph depicting a survival curve showing percent survival of mice.

FIG. 4: illustrates colon length (cm) at sacrifice.

FIG. 5: contains three graphs illustrating OXYRASE and B. subtilis membrane vesicle activity compared via succinate dehydrogenase (SDH) activity and the reduction of O₂.

FIGS. 6A-6B: FIG. 6A shows the percent weight of the mice taken daily, shading represents standard deviation. FIG. 6B shows the percent of initial weight and colon length of mice on day 11.

FIGS. 7A-7B: FIG. 7A shows the percent weight of the mice at day 11 and colon thickness (mg/cm) on day 13 in a Citrobacter induced ulcerative colitis experiment. FIG. 7B shows the CFU/mg of Enterobacteriaceae in mouse stool on day 13.

### DEFINITIONS

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc., which can readily be determined by one of ordinary skill in the art. The term also applies to a dose that induces a particular response in target cells (e.g., the reduction of platelet adhesion and/or cell migration). The specific dose varies depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” or “physiologically compatible” carrier or carrier medium is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition). As used herein, the terms “prevent,” “preventing,” and/or “prevention” may refer to reducing the risk of developing a disease, disorder, or pathological condition.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, or from 0% to 10%, or from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entireties.

The compounds and compositions described herein can be used in methods for treating diseases. In some embodiments, the compounds and compositions described herein can be used in methods for treating diseases by decreasing oxygen levels in the gut.

Some embodiments of the present disclosure may be used to treat gut diseases (e.g., IBD or traveler's diarrhea). In some embodiments, the composition can be taken as a prophylactic for inflammatory bowel disease or traveler's diarrhea; and/or to maintain microbiome diversity. In some embodiments, prophylactic treatment is taken for the purpose of guarding against or preventing the spread or occurrence of disease or infection. In other embodiments, the composition could be taken as a method of treatment during or after a diagnosis of a gut disease.

In some embodiments, microbiome diversity is defined as the amount of individual bacteria from each of the bacterial species present in the gut microbiome. In some embodiments, this microbiome diversity is crucial for maintaining a healthy gut and reducing chance of disease. When there is high richness and diversity of the microbes in the gut, the immune system is stronger and more stable.

The present disclosure relates to depleting the gut of oxygen. In one aspect, bacterial respiration takes place in the cytoplasmic membrane. In some embodiments, introducing membrane vesicles including the cytoplasmic membrane of respiring bacteria into the gut depletes oxygen and improves colon health.

In another aspect, the disclosure provides a method of treating or preventing a disease alleviated by depleting oxygen levels in the gut.

In some embodiments, the disease is a gut inflammatory disease. In some embodiments, the disease is selected from the group consisting of inflammatory bowel disease, traveler's diarrhea, Crohn's disease, ulcerative colitis, celiac disease, polyps, cancer, celiac disease, diverticulitis, malabsorption, short bowel syndrome, gastritis, gastroparesis, gastroenteritis, peptic ulcers, and intestinal ischemia.

Membrane Fragment and/or Vesicles

In one aspect, the present disclosure is directed to a method for removing oxygen from the gut, an anoxic environment. In some embodiments, the method comprises the steps of providing a solution comprising oxygen-scavenging membrane fragments or membrane vesicles to a patient in need thereof. Membrane vesicles are safe and can be derived from bacteria that are GRAS (generally recognized as safe) and that are approved, marketed probiotics.

In some embodiments, the oxygen scavenging membrane fragments can be derived from cytoplasmic membranes the following, but not limited to, Escherichia coli, Salmonella typhimurium, Gluconobacter oxydans, Pseudomonas aeruginosa, Acetobacter, Saccharomyces, Lactobacillus or Bacillus.

In some embodiments, the composition may be in the form of an injection, solution, suspension, or emulsion. In some embodiments, the composition may contain the oxygen scavenging membrane fragments in an amount of about 0.001 units per milliliter to about 100 units per milliliter.

In some embodiments, the oxygen scavenging membrane fragments are stored in a liquid state at sub-zero temperatures to increase their usable lifetime as a pharmaceutical/treatment. In some embodiments, the cryoprotectant may be any compound that decreases the freezing point of water, thereby preventing ice formation or damage. In some embodiments, the composition may contain glycerol or polyethylene glycol as a cryoprotectant. In some embodiments, the composition may contain the cryoprotectant in an amount of about 15% by weight to about 65% by weight, or from about 50 wt % to about 65 wt %.

In some embodiments, a hydrogen donating substance (i.e., an organic substrate) may be necessary in order for the membrane fragments to perform their oxygen removing functions. In some embodiments, suitable hydrogen donors can be, but are not limited to lactic acid, succinic acid, alpha-glycerol phosphate, formic acid, malic acid, pyruvic acid and where available, their corresponding salts. In some embodiments, the hydrogen donating substance may be present in the composition in the amount of about 0.1 wt % to about 5 wt %.

Cryoprotectants are substances used to protect biological tissue from freezing damage. In some embodiments, in order to preserve cells and tissues, cryoprotectants must be non-toxic to the cells. In some embodiments, typical cryoprotectants may include glycerol, dimethyl sulfoxide (DMSO), and glycols, including ethylene glycol, propylene glycol, polyethylene glycol, and glycerol.

In some embodiments, higher concentrations of cryoprotectants (“high” meaning greater than 15 wt %) can be used. In some embodiments, higher concentrations of cryoprotectants prevent the formation of ice crystals, thereby precluding the freezing of cells and the damage and/or killing of preserved cells. In some embodiments, this results in higher numbers of cells being recovered after storage at sub-zero temperatures. In some embodiments, it has been found that cells stored at below zero degrees Celsius are still viable after over twelve (12) months or more of storage.

In some embodiments, the composition comprises glycerol. Glycerol is a small molecule that can pass through the semipermeable membrane of cells and gain entry into their interior. In some embodiments, the cryoprotectant nature of glycerol is available both inside and outside the cell. In some embodiments, glycerol forms strong hydrogen bonds with water molecules, thereby disrupting the crystal lattice formation of ice and maintaining the liquid nature of the composition.

In some embodiments, the composition comprises polyethylene glycol (PEG).

Polyethylene glycol is a cryoprotectant that is impermeable to cell membranes and can be made at different molecular weights. In some embodiments, its mode of action is limited to the space “outside” a cell. In some embodiments, porins (i.e. channels within a cell membrane) allow movement of small hydrophilic molecules between the interior of a cell and the exterior of a cell, large hydrophilic molecules, such as polyethylene glycol, are too large to pass through the porins. In particular embodiments, the polyethylene glycol has about 400 glycol moieties (designated as PEG 400).

In some embodiments, the composition may contain one or more cryoprotectants in the amount of about 15% to about 65% by weight, or from about 50 wt % to about 65 wt %. This lowers the freezing point of the composition. For example, if 53 wt % of glycerol is used, the freezing temperature of the composition is decreased to −26° C.

In some embodiments, the composition may also contain water as a solvent for the various ingredients. In some embodiments, the composition is isotonic. In embodiments, the composition may have an osmolality of about 280 milliOsmoles/liter (mOsm/L) to about 300 mOsm/L.

In this regard, it is contemplated that the effective lifetime of the pharmaceutical composition containing the oxygen scavenging membrane fragments can be extended by storage at sub-zero temperatures. In some embodiments, the composition can thus be stored in a self-defrosting freezer under these conditions. In such freezers, the temperature cycles up and down within a temperature range. For example, the temperature is brought up from −25° C. to −17° C. for about an hour, then reduced to the lower temperature. In some embodiments, this cycle can occur at about 24-hour intervals. Such freezers are significantly cheaper compared to cryogenic freezers. In some embodiments, it is noted that the composition remains in a liquid state at these sub-zero temperatures.

In practical use, after the compositions are warmed to reach room temperature, they are administered to a patient, including a human or other mammal. Thus, it should be noted that the cryoprotectant should be non-toxic to mammals (e.g. humans) at low absolute dosages.

In some embodiments, the dose used in a particular formulation or application is determined by the requirements of the particular disease state and other constraints.

In some embodiments, the pharmaceutical composition may also include other excipients. In some embodiments, particular excipients can include buffering agents, polymers, and stabilizers, which may be useful. In some embodiments, buffering agents are used to control the pH of the composition. In some embodiments, polymers with nonpolar moieties such as polyethylene glycol can also be used as surfactants. In some embodiments, protein stabilizers can include polyols, sugars, amino acids, amines, and salts. In some embodiments, suitable sugars include sucrose and trehalose. In some embodiments, amino acids include histidine, arginine, glycine, methionine, proline, lysine, glutamic acid, and mixtures thereof. It should be noted that particular molecules can serve multiple purposes. For example, in some embodiments, histidine can act as a buffering agent and an antioxidant.

In some embodiments, the present disclosure removes oxygen through the use of oxygen scavenging membrane fragments. In some embodiments, the membrane fragments, which contain an electron transport system that reduces oxygen to water, may be obtained from various sources. It is known that a great number of organisms have cytoplasmic membranes which contain the electron transport system that effectively reduces oxygen to water if a suitable hydrogen donor is present in the medium. In some embodiments, some suitable bacterial sources include, but are not limited to, Escherichia coli, Salmonella typhimurium, Gluconobacter oxydans, Pseudomonas aeruginosa, Acetobacter, Lactobacillus and Bacillus. In some embodiments, bacterial membranes have been highly effective in removing oxygen from media and other aqueous and semi-solid environments.

In some embodiments, the same oxygen reducing effects produced by the cell membrane fragments from the bacteria sources indicated above can also be obtained by the use of oxygen reducing membranes from, for example, the mitochondrial organelles of a large number of higher non-bacteria organisms. More particularly, a great number of fungi, yeasts, plants, and animals have mitochondria that reduce oxygen to water if a suitable hydrogen donor is present in the medium. In some embodiments, sources of oxygen reducing membranes from these mitochondria are: beef heart muscle, potato tuber, spinach, Saccharomyces, Neurospora, Aspergillus, Euglena, and Chlamydomonas.

In some embodiments, the exact amount of membranes containing the enzyme systems needed to reduce oxygen in the gut can vary by a number of parameters including pH, temperature, kinds and amounts of substrate present, and amount of oxygen present within the tumor. In some embodiments, the pharmaceutical composition contains the oxygen scavenging membrane fragments in the amount of about 0.001 units/mL to about 100 units/mL, or from about 0.01 units/mL to about 10 units/mL, or from about 0.3 unit/mL to about 10 units/mL, or from about 1 unit/mL to about 10 units/m L.

In some embodiments, the composition may contain the oxygen scavenging membrane fragments in an amount of about 0.001 units per milliliter (u/mL) to about 100 units per milliliter. In some embodiments, the composition may contain the oxygen scavenging membrane fragments in an amount of greater than 0.1 units per milliliter, or in an amount of at least 0.5 units per milliliter, or in an amount of at least 5 units per milliliter.

In some embodiments, the pharmaceutical composition may include a pharmaceutically acceptable carrier, which acts as a vehicle for delivering the membrane fragments. In some embodiments, examples of pharmaceutically acceptable carriers include, but are not limited to, liquid carriers like water, oil, and alcohols, in which the molecular antagonists can be dissolved or suspended and microspheres.

In one embodiment, the membrane vesicles are administered intracolonically.

In some embodiments, the membrane vesicles aid with other dysbiotic states (e.g. antibiotic induced dysbiosis). In some embodiments, membrane vesicles are used to aid therapeutic interventions (e.g. fecal microbiota transplants).

### Oxyrase

OXYRASE is a commercially available enzyme system (from Oxyrase, Inc.) obtained from the cytoplasmic membranes of Escherichia coli to produce anaerobic conditions in a wide variety of environments. In some embodiments, the system is available in the form of membrane fragments that scavenge oxygen.

Some embodiments of the present disclosure may use commercially available membrane vesicles from Escherichia coli (E. coli), available as OXYRASE, to remove oxygen in the gut and prevent blooms of Enterobacteriaceae and other undesirable bacteria. OXYRASE was originally designed for in vitro removal of oxygen from growth media. In some embodiments, OXYRASE supplementation in mouse chow improves disease outcomes in a chemically induced model of colitis in mice, likely by reduction of oxygen levels in the gut. In some embodiments, compositions in accordance with the disclosure can be taken as a parabiotic to prevent inflammatory bowel disease development; can be derived as parts of organisms that are GRAS, yielding a good safety profile; and can be used to modulate the microbiome without off-target effects like the current standard of care (5′-ASA).

In some embodiments, a parabiotic is defined as a compound that induces the growth or activity of beneficial microorganisms such as bacteria and fungi. In some embodiments, parabiotics can alter the composition of organisms in the gut microbiome.

In some embodiments, OXYRASE consists of an enzyme system derived from the cytoplasmic membranes of microorganisms. In some embodiments, sterile (EC) and nonsterile (EC/NS) OXYRASE in particular are derived from the cell membrane fragments of E. coli (0.2 microns or smaller) suspended in 20 mM phosphate buffer at a neutral pH. In some embodiments, substrates for OXYRASE may include lactic acid, succinic acid, formic acid, or their salts, and alpha-glycerol phosphate in addition to oxygen. In some embodiments, one unit/ml OXYRASE activity reduces dissolved oxygen (air saturated 40 mM phosphate buffer, pH 8.4, at 37 degrees Celsius) at the rate of 1% per second. In some embodiments, the rate of oxygen removal increases with temperature, and above 55 degrees Celsius, OXYRASE begins to be inactivated but persists up to 80 degrees Celsius. In some embodiments, OXYRASE is active over a wide pH range of 6.8 to 8.4.

### Pharmaceutical Compositions

Described below are non-limiting pharmaceutical compositions and methods for preparing the same.

### Pharmaceutical Compositions for Oral Administration

In an embodiment, the disclosure provides a pharmaceutical composition for oral administration containing the active pharmaceutical ingredient or combination of active pharmaceutical ingredients, such as the composition described herein, and a pharmaceutical excipient suitable for oral administration.

In some embodiments, the disclosure provides a solid pharmaceutical composition for oral administration containing: (i) an effective amount of an active pharmaceutical ingredient or combination of active pharmaceutical ingredients, and (ii) a pharmaceutical excipient suitable for oral administration. In selected embodiments, the composition further contains (iii) an effective amount of a third active pharmaceutical ingredient, and optionally (iv) an effective amount of a fourth active pharmaceutical ingredient.

In some embodiments, the pharmaceutical composition may be a liquid pharmaceutical composition suitable for oral consumption. Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl-lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogs thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Other Pharmaceutical Compositions

Pharmaceutical compositions described herein may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of an active pharmaceutical ingredient or combination of active pharmaceutical ingredients or a pharmaceutical composition thereof can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The active pharmaceutical ingredient or combination of active pharmaceutical ingredients can also be administered intradiscally or intrathecally.

Exemplary parenteral administration forms include solutions or suspensions of active compound in sterile aqueous solutions, for example, aqueous propylene glycol or dextrose solutions. Such dosage forms can be suitably buffered, if desired.

### Dosages and Dosing Regimens

The amounts of the pharmaceutical compositions administered using the methods herein, such as the dosages of the present composition, is dependent on the human or mammal being treated, the severity of the disorder or condition, the rate of administration, the disposition of the active pharmaceutical ingredients and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of the pharmaceutical compositions and active pharmaceutical ingredients may be provided in units of mg/kg of body mass or in mg/m² of body surface area.

In some embodiments, a pharmaceutical composition or active pharmaceutical ingredient is administered in a single dose. Such administration may be by injection, e.g., intravenous injection, in order to introduce the active pharmaceutical ingredient quickly. However, other routes, including the preferred oral route, may be used as appropriate. A single dose of a pharmaceutical composition may also be used for treatment of an acute condition.

In some embodiments, a pharmaceutical composition or active pharmaceutical ingredient is administered in multiple doses. In an embodiment, a pharmaceutical composition is administered in multiple doses. Dosing may be once, twice, three times, four times, five times, six times, or more than six times per day. Dosing may be once a month, once every two weeks, once a week, or once every other day. In other embodiments, a pharmaceutical composition is administered about once per day to about 6 times per day. In some embodiments, a pharmaceutical composition is administered once daily, while in other embodiments, a pharmaceutical composition is administered twice daily, and in other embodiments a pharmaceutical composition is administered three times daily.

Administration of the active pharmaceutical ingredients may continue as long as necessary. In selected embodiments, a pharmaceutical composition is administered for more than 1, 2, 3, 4, 5, 6, 7, 14, or 28 day(s). In some embodiments, a pharmaceutical composition is administered for less than 28, 14, 7, 6, 5, 4, 3, 2, or 1 day(s). In some embodiments, a pharmaceutical composition is administered chronically on an ongoing basis—e.g., for the treatment of chronic effects. In some embodiments, the administration of a pharmaceutical composition continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of an active pharmaceutical ingredient disclosed herein, for example the present composition, is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of an active pharmaceutical ingredient disclosed herein is less than about 25 mg, less than about 50 mg, less than about 75 mg, less than about 100 mg, less than about 125 mg, less than about 150 mg, less than about 175 mg, less than about 200 mg, less than about 225 mg, or less than about 250 mg. In some embodiments, an effective dosage of an active pharmaceutical ingredient disclosed herein is greater than about 25 mg, greater than about 50 mg, greater than about 75 mg, greater than about 100 mg, greater than about 125 mg, greater than about 150 mg, greater than about 175 mg, greater than about 200 mg, greater than about 225 mg, or greater than about 250 mg.

In some embodiments, an effective dosage of an active pharmaceutical ingredient disclosed herein, for example the present composition, is in the range of about 0.01 mg/kg to about 200 mg/kg, or about 0.1 to 100 mg/kg, or about 1 to 50 mg/kg.

In some embodiments, an active pharmaceutical ingredient is administered at a dosage of 10 to 200 mg BID, including 50, 60, 70, 80, 90, 100, 150, or 200 mg BID. In some embodiments, an active pharmaceutical ingredient is administered at a dosage of 10 to 500 mg BID, including 1, 5, 10, 15, 25, 50, 75, 100, 150, 200, 300, 400, or 500 mg BID.

In some instances, dosage levels below the lower limit of the aforesaid ranges may be more than adequate, while in other cases still larger doses may be employed without causing any harmful side effect, e.g., by dividing such larger doses into several small doses for administration throughout the day. Of course, as those skilled in the art appreciate, the dosage actually administered depends upon the condition being treated, the age, health and weight of the recipient, the type of concurrent treatment, if any, and the frequency of treatment. Moreover, the effective dosage amount may be determined by one skilled in the art on the basis of routine empirical activity testing to measure the bioactivity of the compound(s) in a bioassay, and thus establish the appropriate dosage to be administered.

An effective amount of the combination of the active pharmaceutical ingredient may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

In some embodiments, the compositions described herein further include controlled-release, sustained release, or extended-release therapeutic dosage forms for administration of the compounds described herein, which involves incorporation of the compounds into a suitable delivery system in the formation of certain compositions. This dosage form controls release of the compound(s) in such a manner that an effective concentration of the compound(s) in the bloodstream may be maintained over an extended period of time, with the concentration in the blood remaining relatively constant, to improve therapeutic results and/or minimize side effects. Additionally, a controlled-release system would provide minimum peak to trough fluctuations in blood plasma levels of the compound.

The following examples describe the disclosure in further detail. These examples are provided for illustrative purposes only and should in no way be considered as limiting the disclosure.

### EXAMPLES

Described below are non-limiting examples for reducing oxygen levels in the gut.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it is apparent to those of ordinary skill in the art that other embodiments incorporating the concepts disclosed herein may be used without departing from the spirit and scope of the invention. Accordingly, the described embodiments are to be considered in all respects as only illustrative and not restrictive.

### Example 1: Efficacy of Oxyrase in Mouse Model

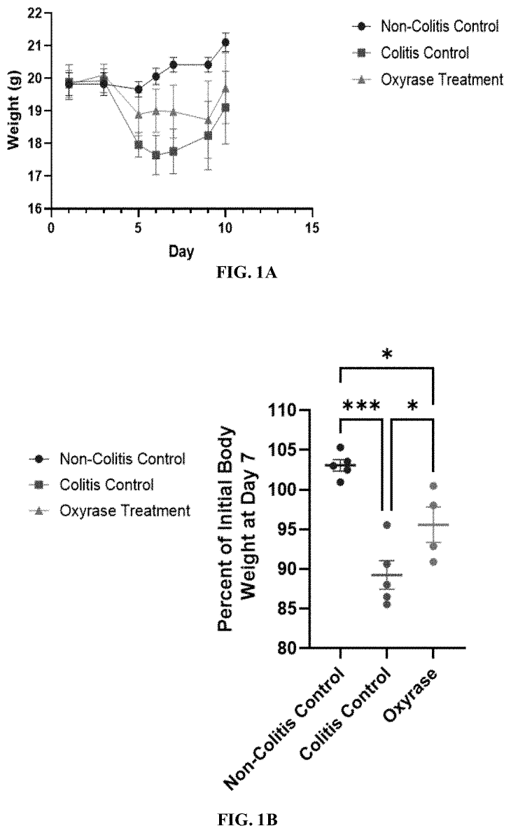

The efficacy of OXYRASE was tested in a pilot experiment in a dextran sodium sulfate (DSS) mouse model of colitis. The DSS mouse model of colitis is a widely used model of inflammatory bowel disease most closely resembling ulcerative colitis. Eight-week old C57/BL6 mice were given 2.5% DSS (M.W. 40,000 Da) (colitis control and OXYRASE treatment) in drinking water for six days to induce colitis; on day four, mice were orally inoculated with 10⁹ cells of Escherichia coli AR350 in LB broth to promote inflammation and an Enterobacteriaceae bloom. Mice were given control, normal chow (colitis control, non-colitis control) or mouse chow supplemented with OXYRASE (OXYRASE treatment) in a 1:12 ratio (OXYRASE weight:chow volume) for the duration of the experiment. OXYRASE treatment prevented weight loss compared to the colitis control mice (FIG. 1A); on day 7, 24 hours after DSS removal, the percent of initial weight was significantly higher in mice treated with OXYRASE compared to the colitis control (FIG. 1B).

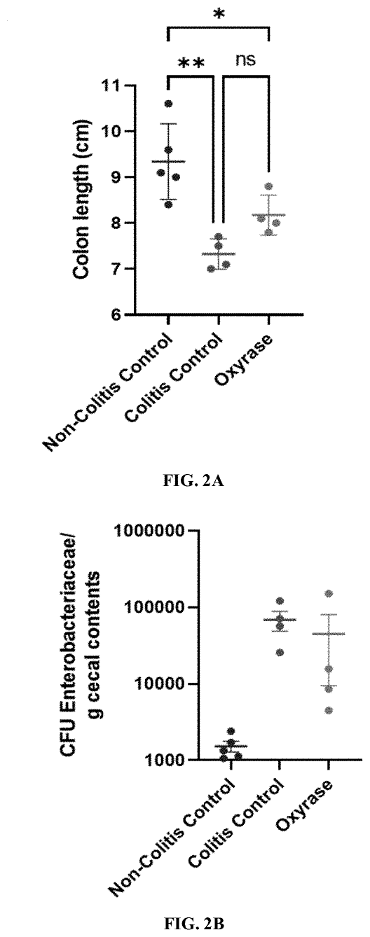

Furthermore, upon sacrifice, mice that received OXYRASE tended to have longer colon lengths (FIG. 2A) and a lower absolute abundance of Enterobacteriaceae in cecal contents (FIG. 2B). Based on the efficacy of OXYRASE in improving colitis symptoms in this experiment the ability of OXYRASE was tested to ameliorate more severe colitis.

### Example 2: Improved Survival in a Severe DSS Colitis Mouse Model

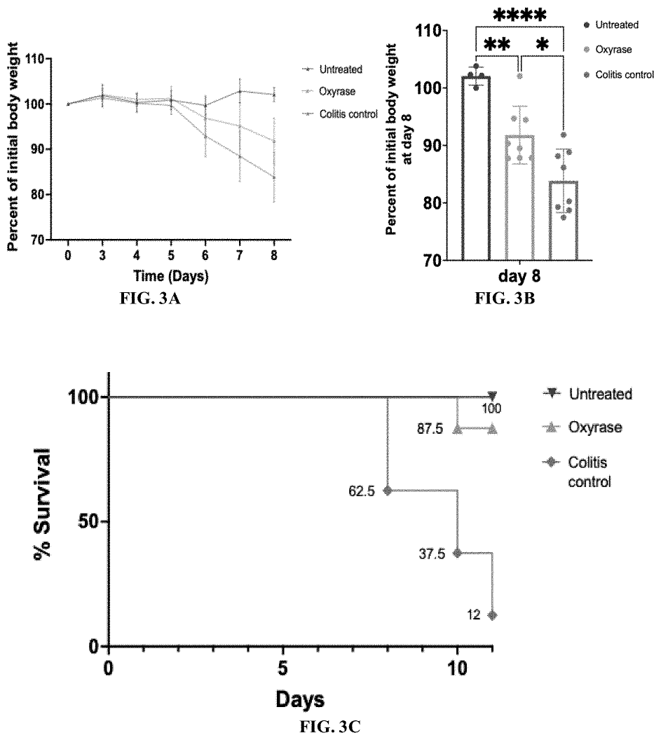

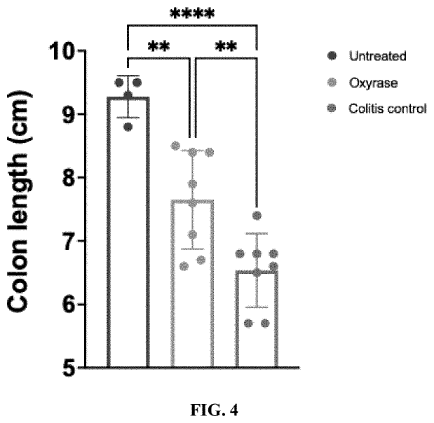

The ability of OXYRASE to alleviate colitis was further tested in a severe colitis model. In this experiment the mice were subjected to an increased concentration of DSS at 3%, all other experimental parameters remained the same. In a severe colitis model OXYRASE again reduced weight loss over the course of the experiment when compared to the colitis control (FIG. 3A) and mice treated with OXYRASE had significantly less weight loss on day 8 than the colitis control (FIG. 3B). OXYRASE also improved the survival of mice given 3% DSS based upon a humane endpoint wherein mice that lost 20% or more of their initial body weight were sacrificed. Using this criteria OXYRASE increased the survival of mice given 3% DSS by 75.5% (FIG. 3C). Mice with severe colitis treated with OXYRASE had significantly longer colons than the untreated colitis control (FIG. 4). Taken together, the results of these colitis experiments suggest that OXYRASE improves survival and colon integrity in an acute, chemical mouse model of colitis.

### Example 3: Membrane Vesicles (MVs) from the Bacterium Bacillus subtilis

Having shown that Oxyrase can be effective in ameliorating colitis it was sought to improve the efficacy of the treatment by using membrane vesicles made from Bacillus subtilis. Because Oxyrase is made from E. coli and E. coli associated LPS can have proinflammatory effects that exacerbate colitis, membrane vesicles were made from the gram positive GRAS bacterium Bacillus subtilis which does not have LPS and should not exacerbate colitis. Bacillus subtilis membrane vesicles were tested in a DSS and E. coli AR350 induced ulcerative colitis mouse model against Oxyrase. When normalizing membrane vesicles by activity it was observed that the B. subtilis MVs had better protective effects than Oxyrase in this experiment likely because the B. subtilis MVs lacked the antigen LPS.

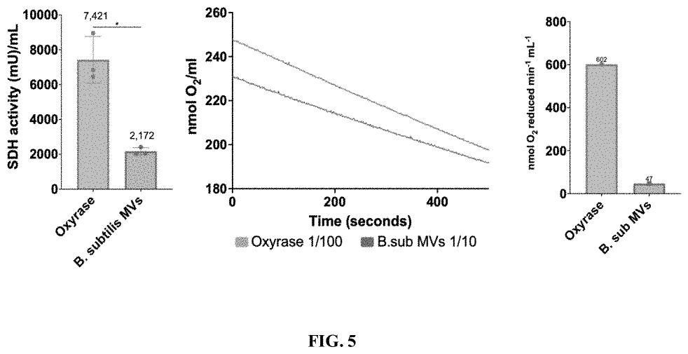

The activity of membrane vesicles was compared via succinate dehydrogenase (SDH) activity and the reduction of O₂. SDH was measured with a colorimetric assay and O₂ concentration was measured with a Clark electrode. In FIG. 5, bars represent the mean plus or minus the standard deviation. Statistical significance was determined using Welch's t test. *, P-value<0.05.

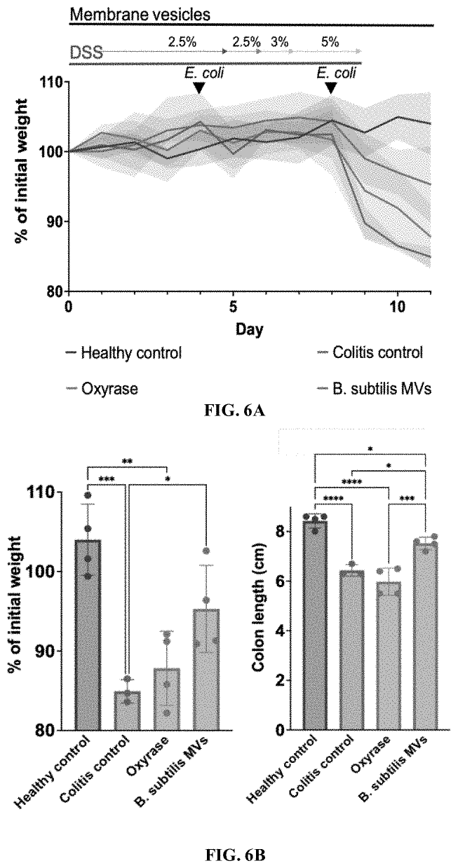

Eight-week old C57BL/6 mice were given between 2.5-5% DSS (M.W. 40,000 Da) (colitis control, OXYRASE treatment, and B. subtilis MVs) in drinking water for nine days to induce colitis; on day four and day 8, mice were orally inoculated with 10⁹ cells of Escherichia coli AR350 in LB broth (colitis control, OXYRASE treatment, and B. subtilis MVs). Mice were given normal chow (colitis control, non-colitis control) or mouse chow supplemented with OXYRASE or B. subtilis MVs for 11 days at either 1:61 of OXYRASE or 1:18 of B. subtilis MVs to mouse chow volume to weight. For all groups n=4. FIG. 6A shows the percent weight of the mice taken daily, shading represents standard deviation. FIG. 6B shows the % of initial weight and colon length of mice on day 11. Bars represent the mean plus or minus the standard deviation. Statistical significance was determined using Tukey's multiple comparison test. *, P value<0.05, **, P value<0.005, ***, P-value<0.0005, ****, P-value of <0.0001.

### Example 4: Vehicle Control and Citrobacter Induced Colitis

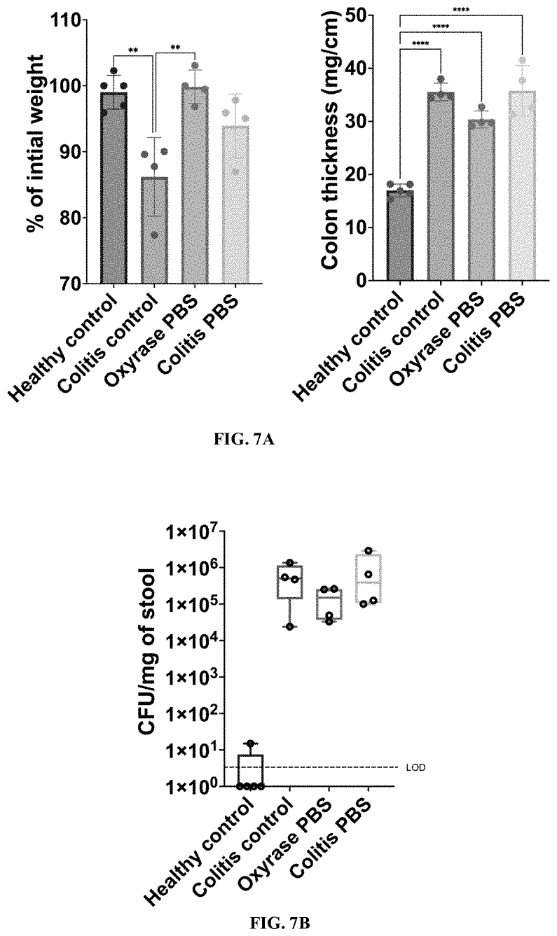

FIGS. 7A and 7B: To further strengthen the claim that membrane vesicles can ameliorate colitis Oxyrase was tested in a separate model of Citrobacter induced ulcerative colitis. Citrobacter rodentium is a natural pathogen of mice, a member of the Enterobacteriaceae family and it shares several virulence factors with enterohemorrhagic and enteropathogenic E. coli responsible for human gastrointestinal infection making it an ideal pathogen to study bacterial induced ulcerative colitis in mice. Additionally, to demonstrate that the protective effects of membrane vesicles were not due to buffer a vehicle control feeding the mice PBS was included in their food.

Eight-week old C57BL/6 mice were given Oxyrase washed in PBS (Oxyrase PBS) or a PBS control (PBS colitis) 1:12 volume to weight of food on day 0, colitis control mice and healthy control mice were given normal mouse chow. On Day 1 colitis control, Oxyrase PBS and PBS colitis mice were orally gavaged with 1×10⁸ cells of Citrobacter rodentium in PBS. For colitis control, Oxyrase PBS and colitis PBS n=4 for healthy control n=5. FIG. 7A: percent of initial weight of mice at day 11 of the experiment and colon thickness at day 13 when the mice were sacrificed. Increasing colon thickness and weight loss are common symptoms associated with ulcerative colitis. Colon thickness is calculated as the weight of the colon divided by its length. Bars represent the mean plus or minus the standard deviation. Statistical significance was determined using Tukey's multiple comparison test.**, P value<0.005, ****, P-value of <0.0001. FIG. 7B: colony forming units of Enterobacteriaceae per milligram of stool. Box displays lower and upper quartile, line indicates median, bars represent maximum and minimum values. Oxyrase on average, mitigates weight loss, decreases colon thickness and decreases the absolute abundance of disease causing Enterobacteriaceae when compared to the colitis control and vehicle control. These data further indicate that membrane vesicles alone can ameliorate ulcerative colitis.

What is claimed is:

1. A method of treating a disease or condition associated with gut inflammation comprising administering, to a patient in need thereof, a therapeutically effective amount of a composition comprising oxygen-scavenging membrane fragments and/or membrane vesicles, or a pharmaceutically acceptable salt, solvate, hydrate, cocrystal, or prodrug thereof.

2. The method of claim 1, wherein the oxygen-scavenging membrane fragments and/or membrane vesicles are derived from bacteria.

3. The method of claim 1, wherein the oxygen-scavenging membrane fragments and/or membrane vesicles are derived from cytoplasmic membranes of Escherichia coli, Salmonella typhimurium, Gluconobacter oxydans, Pseudomonas aeruginosa, or Acetobacter.

4. The method of claim 1, wherein sources of the oxygen-scavenging membranes are beef heart muscle, potato tuber, spinach, Saccharomyces, Neurospora, Aspergillus, Euglena, or Chlamydomonas.

5. The method of claim 1, wherein the composition contains the oxygen-scavenging membrane fragments in the amount of about 0.001 units/mL to about 100 units/mL, from about 0.01 units/mL to about 10 units/mL, from about 0.3 unit/mL to about 10 units/mL, or from about 1 unit/mL to about 10 units/mL.

6. The method of claim 1, wherein the composition is administered as a parabiotic to prevent inflammatory bowel disease development.

7. The method of claim 1, wherein the composition is taken to maintain microbiome diversity.

8. The method of claim 1, wherein the composition is OXYRASE.

9. The method of claim 1, wherein the disease or condition is inflammatory bowel disease, traveler's diarrhea, Crohn's disease, ulcerative colitis, celiac disease, polyps, cancer, celiac disease, diverticulitis, malabsorption, short bowel syndrome, gastritis, gastroparesis, gastroenteritis, peptic ulcers, or intestinal ischemia.

10. The method of claim 1, wherein the composition contains a cryoprotectant in an amount of about 15% by weight to about 65% by weight, or from about 50 wt % to about 65 wt %.

11. The method of claim 10, wherein the cryoprotectant is s glycerol, dimethyl sulfoxide (DMSO), and glycols, including ethylene glycol, propylene glycol, polyethylene glycol, or glycerol.

12. The method of claim 1, wherein the composition further comprises a hydrogen donating substance in the amount of about 0.1 wt % to about 5 wt %.

13. The method of claim 12, wherein the oxygen-scavenging membrane fragments and/or membrane vesicles are derived from mitochondria that reduce oxygen to water in the presence of the hydrogen donating substance.

14. The method of claim 12, wherein the donating substance is lactic acid, succinic acid, alpha-glycerol phosphate, formic acid, malic acid, pyruvic acid or the corresponding salts thereof.

15. The method of claim 1, wherein the composition may be in the form of an injection, a solution, a suspension, or emulsion.

16. The method of claim 1, wherein the composition is taken orally.

17. The method of claim 1, wherein the composition is administered through a rectal route.

18. The method of claim 1, wherein the composition is administered intracolonically.
